(19) United States
(12) Patent Application Publication
(10) Pub. No.: US 2010/0268190 A1
(43) Pub. Date: Oct. 21, 2010

(54) Medical device to be used by a patient

(75) Inventor: Holger Mielenz, Ostfildern (DE)

Correspondence Address: KENYON & KENYON LLP, ONE BROADWAY, NEW YORK, NY, 10004, US

(73) Assignee: Robert Bosch GmbH, Stuttgart (DE)

(21) Appl. No.: 12/798,692

(22) Filed: Apr. 9, 2010

(30) Foreign Application Priority Data
Apr. 21, 2009 (DE) 10 2009 002 537.5

(51) Int. Cl. A61M 5/20

(52) U.S. Cl. 604/506; 604/131

(57) ABSTRACT

A medical device to be used by a patient, having release means for administering a drug, in particular subcutaneously, and lockable triggering means for triggering the administration of the drug, characterized by telecommunication means, which are connected to the lockable triggering means in order to activate the triggering means in response to the reception data from a remote unit. The device according to the invention in particular assists in the self-injection of therapeutic agents by a patient and reduces the risk of an improper application.

### CROSS-REFERENCE TO RELATED APPLICATIONS

This application claims the benefit under 35 U.S.C. §119 of Germany priority application no. 10 2009 002 537.5 filed on Apr. 21, 2009, the contents of which are expressly incorporated herein by reference.

### BACKGROUND OF THE INVENTION

1. Field of the Invention

The present invention relates to a medical device to be used by a patient and to a system comprising at least one such medical device.

2. Description of Related Art

Today, in addition to simple tablet or ointment products, many patients must also self-administer drugs by injections, mainly subcutaneous injections. The self-application of drugs by injection is now used in many diseases, which usually require multiple administrations per week. These include e.g. drugs for adjusting the coagulation system, for stabilizing auto-immune diseases such as e.g. multiple sclerosis (MS), chronic metabolic diseases or diseases caused by old age.

Because of the continuing development of new drugs there is also a rise in the number of drugs that for reasons of bioavailability and pharmacokinetics can only be administered by injection. To assist patients in the application of the therapeutic agents, pharmaceutical manufacturers are increasingly developing application sets that to a certain degree reveal an automation in the injection. For this purpose, the preparation of the injection solution and the application of the device to the injection point are left to the patient. The functionality of today's self-application machines describes merely the automated insertion of the injection needle into the skin and a standardized administration of the drug via a defined inflow speed. This process is triggered by pressing a button.

The rising number of self-applications results from the fact that increasingly fewer services of a general practitioner may be settled via the health insurance companies and that hence the tendency grows toward application sets that are easy to operate.

Increasingly, the patient is thus personally responsible also for his medication in the case of injections and for the purpose of performing this task usually receives a one-time introduction into the operation and is subsequently left to his own devices. For many patients this presents a great challenge, and frequently such patients are unable to manage the new task comfortably. Older people in particular have great inhibitions in handling application sets and quickly become insecure.

One false application of the application sets may on the one hand reduce the effect of the drug because of a partial loss of specimen or an injection into the wrong tissue (periosteum or nerve instead of muscle). On the other hand, the damage to the mentioned organs such as the periosteum, nerves and blood vessels cause pain in the patient. At the same time, the mechanical effects of the needle and/or the wrongly applied drug may result in infections, which may also take chronic courses. An example of this is an injection into the buttocks, where frequently the pelvic bone or the sciatic nerve is hit instead of the muscle tissue, which may result in the development of painful infections.

In this case there is the danger that, in reaction to the lack of effect and/or the experienced pain, the patient refrains from using the drug further.

It is thus desirable to indicate a medical device that simplifies the administration of a drug for the patient.

### SUMMARY OF THE INVENTION

This and other objects of the invention are achieved by a medical device to be used by a patient and by a system for monitoring the, in particular subcutaneous, release of a drug. The medical device has release means (210) for administering a drug, in particular subcutaneously, and lockable triggering means (220) for triggering the administration of the drug, characterized by telecommunication means (230), which are connected to the lockable triggering means (220), for activating the triggering means in response to the reception of data from a remote unit (240, 300).

The present invention is represented schematically in the drawing based on an exemplary embodiment and is described in detail below with reference to the drawing.

### BRIEF DESCRIPTION OF THE SEVERAL VIEWS OF THE DRAWING

FIG. 1 schematically shows a preferred specific embodiment of a system according to the present invention for monitoring the subcutaneous release of a drug to a patient.

### DETAILED DESCRIPTION OF THE INVENTION

The device according to the present invention in particular assists in the self-injection of therapeutic agents by a patient and reduces the risk of an improper application of the injection needle. A targeted application of the injection needle prevents physically and psychologically caused consequential damage in the patient such as e.g. infections or chronic diseases, which often manifest themselves as a result of an improper handling of the injection instruments and/or a local misapplication of the drug.

The medical device of the present invention, which is to be operated or handled by a patient, has release means for administering a drug, in particular subcutaneously. The release means may comprise in particular a needle, a container for receiving the drug (for example an ampoule), which are also exchangeable individually or jointly. For example, the release means may be developed to receive an element, which includes a needle as well as a container containing the drug. The device furthermore has lockable triggering means for triggering the administration of the drug. The triggering means may be developed in particular as a button or trigger and be lockable particularly electronically in order to prevent an unintended or improper administration of the drug. The device furthermore has telecommunication means, which are connected to the lockable triggering means in order to activate the triggering means in response to the reception of data.

Preferably, recording means are provided for recording image and/or sound data which are transmitted to the remote unit. Just as advantageously, reproduction means are provided for reproducing image and/or sound data which are received from the remote unit. Thus it is possible to assist the patient for example in finding a suitable treatment point or injection point. For this purpose, a telemedical service based e.g. on Internet technology is provided, which readily allows the patient to communicate bidirectionally with an expert, e.g. a physician or specially trained nursing staff. In this connection, the expert personnel, also called operator, and the patient should be given the option of communicating visually and auditorily. In a simple embodiment, this could be accomplished by Web cameras and corresponding microphones, which is possible via Internet portals for example. Depending on the requirements of the respective drug, the operator is thus able to give the patient instructions for finding the possible injection point. This assistance is independent of the utilized administration device.

The telecommunication means are preferably connected to the lockable triggering means in order to activate or enable the triggering means upon reception of enabling data so that the patient can operate them. In particular in combination with the recording means and reproduction means, this embodiment displays particular advantages. The image and/or sound data may be transmitted to a remote unit for example and be reproduced there, where they may be inspected by an operator, for example a physician or medical specialists. If the operator detects that the medical device was properly placed, then he is able to unlock the triggering means by transmitting enabling data in order to allow the patient to administer the drug. The patient is then able to actuate or trigger the triggering means in order to administer the drug to himself.

In a refinement, the telecommunication means are connected to lockable triggering means in order to trigger the triggering means in response to the reception of triggering data. With reference to the just explained remote unit, the operator may transmit triggering data in order to trigger the triggering means and thus the administration of the drug if a proper placement of the medical device is detected.

Known devices for the self-application of injections offer no possibility for detecting and/or plausibilizing a correct injection point. The preferred refinement of the present invention includes the possibility of telecommunication for plausibilizing the injection point. As a result, the risk of a misapplication on the part of the user is markedly reduced. At the same time, the risk of a painful application of the drugs is reduced, which creates trust on the part of the patient and markedly increases the chances of a regular application of the drug with full efficiency. The direct and always available contact to an operator is significant as a special motivator at the psychological level in particular for patients who have great inhibitions in handling medical devices. Additional advantages are the possibility of supervision on the part of the operator over the regularity of the administration of drug or the possibility of providing assistance in preparing the injection solution. Usually, drugs in solution do not remain stable for long, which is why the patient must only be bring them into solution just prior to application. This process is usually difficult for older persons.

Advantageously, the medical device is equipped with means for detecting the drug to be administered. Sensors such as e.g. an RFID scanner are suitable for this purpose. Particularly in combination with suitably equipped ampoules or sets (needle and container), which have RFID tags, is it possible to detect the kind of drug in the medical device. For example, by transmitting the detected data to the remote unit, an operator is able to verify whether the medical device was filled with the correct drug. Only following a verification is the operator able to transmit the appropriate data to the telecommunication means in order to activate the triggering means.

The device is expediently set up to transmit data regarding the kind of drug, the administered quantity and/or the time of the administration to the remote unit after the drug has been administered. These data in particular may be stored at the remote unit and held ready for later evaluation purposes. Alternatively or additionally, the audio and visual data streams are stored and archived for documentation purposes. The control signals legitimated by the specialist personnel (e.g. triggering and/or enabling an injector) may be handled in the same way.

Additional advantages and refinements of the present invention derive from the description and the accompanying drawing.

It is understood that the features mentioned above and yet to be described below may be used not only in the respectively indicated combination, but also in other combinations, or alone, without departing from the scope of the present invention.

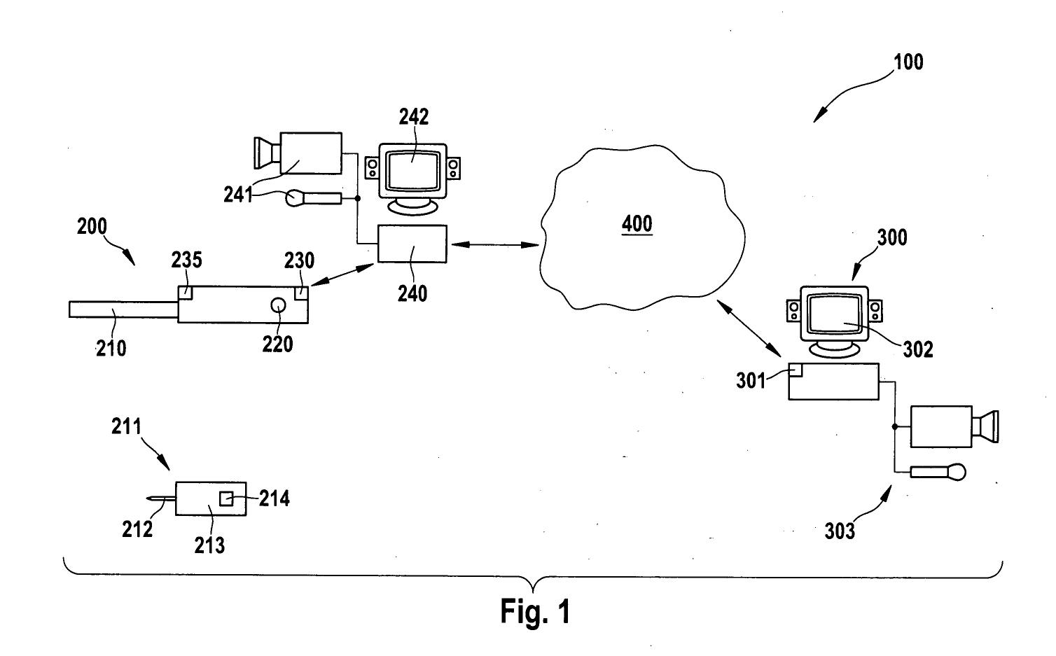

FIG. 1 schematically shows a preferred embodiment of a system according to the present invention for monitoring the subcutaneous release of a drug to a patient, which is designated as a whole by 100.

System 100 has a preferred embodiment of a medical device 200, to be operated or handled by a patient, and a remote unit developed in this case as a computer 300. Medical device 200 and computer 300 are connected via a telecommunication network 400, for example the Internet. Depending on the design of the telecommunication network and the possible access to it by third parties, encryption and/or authentication methods according to the related art are provided in order to secure the communication between the medical device and the remote unit.

The device has telecommunication means 230 for connecting to network 400. The telecommunication means may be developed to be wired or wireless (WLAN, UMTS, radio etc.). The telecommunication means may establish a connection to telecommunication network 400 directly or, as presently shown, indirectly via an interposed PC 240. It is possible that an appropriate transmission software runs on PC 240 for example.

PC 300 has telecommunication means 301 for receiving data from device 200 and for transmitting data to device 200.

Medical device 200 has release means 210 for the subcutaneous administration of a drug. Release means 210 are developed to accommodate individual elements 211, which have a needle 212 and a container 213 containing the drug. According to the preferred specific embodiment shown here, elements 211 also have an indicator 214 in order to communicate to device 200 the kind of drug and optionally its concentration. Indicator 214 may be developed, for example, as an RFID tag. Device 200 accordingly has detection means, developed here as RFID scanner 235, for detecting or recognizing the drug.

Device 200 furthermore has lockable triggering means, developed here as trigger button 220, for triggering the subcutaneous administration of the drug. The triggering means are connected to telecommunication means 230.

The system furthermore has recording means 241 for recording image and/or sound data, which in this case are connected as camera and microphone to PC 240. The device here too has reproduction means 242 developed as a screen having loudspeakers for reproducing image and/or sound data for the patient that were recorded and/or received from PC 300. An appropriate recording and reproduction software may be running on PC 240. It is practical to integrate the image and sound recording into the software for data transmission.

The remote unit developed as PC 300 also has reproduction means 302 developed as screen and loudspeakers for reproducing recorded and/or received image and/or sound data for an operator. PC 300 additionally has recording means 303 developed as camera and microphone for recording image and/or sound data to be transmitted to the patient.

It is understood that the represented figures show only an exemplary specific embodiment of the present invention. In addition, any other specific embodiment is conceivable, without departing from the scope of this invention.

What is claimed is:

1. A medical device to be used by a patient, comprising: release means for administering a drug, lockable triggering means for triggering the administration of the drug, and telecommunication means, which are connected to the lockable triggering means, for activating the triggering means in response to reception of data from a remote unit.

2. The medical device as recited in claim 1, wherein the telecommunication means is connected to the lockable triggering means in order to enable the triggering means in response to the reception of enabling data so that the patient is able to operate them.

3. The medical device as recited in claim 1, wherein the telecommunication means is connected to the lockable triggering means in order to trigger the triggering means in response to the reception of triggering data.

4. The medical device as recited in claim 1, further comprising detection means for detecting or recognizing the drug to be administered.

5. The medical device as recited in claim 2, further comprising detection means for detecting or recognizing the drug to be administered.

6. The medical device as recited in claim 3, further comprising detection means for detecting or recognizing the drug to be administered.

7. The medical device as recited in claim 1, which is able to transmit data regarding at least one of: the kind of drug, the administered quantity and the time of administration to the remote unit, after the drug has been administered.

8. The medical device as recited in claim 1, further comprising recording means for recording at least one of image and sound data, which are transmitted to the remote unit.

9. The medical device as recited in claim 2, further comprising recording means for recording at least one of image and sound data, which are transmitted to the remote unit.

10. The medical device as recited in claim 3, further comprising recording means for recording at least one of image and sound data, which are transmitted to the remote unit.

11. The medical device as recited in claim 4, further comprising recording means for recording at least one of image and sound data, which are transmitted to the remote unit.

12. The medical device as recited in claim 1, further comprising reproduction means for reproducing at least one of image and sound data, which are received from the remote unit.

13. The medical device as recited in claim 2, further comprising reproduction means for reproducing at least one of image and sound data, which are received from the remote unit.

14. The medical device as recited in claim 3, further comprising reproduction means for reproducing at least one of image and sound data, which are received from the remote unit.

15. The medical device as recited in claim 1, wherein the release means is capable of administering the drug subcutaneously.

16. A system for monitoring the administration of a drug to a patient, comprising: at least one medical device as recited in claim 1, and a remote unit, the remote unit having telecommunication means for receiving data from the at least one medical device and for transmitting data to the at least one medical device.

17. The system as recited in claim 16, further comprising reproduction means for reproducing at least one of image and sound data, which are received from the at least one medical device.

18. The system as recited in claim 16, further comprising recording means for recording at least one of image and sound data, which are transmitted to the at least one medical device.

19. A method for monitoring the subcutaneous administration of a drug to a patient, the patient being connected to an operator by the use of telecommunication means and the operator telecommunicationally defining an administration point for administering the drug using a medical device as recited in claim 1.
